(19) United States
(12) Patent Application Publication
(10) Pub. No.: US 2014/0116441 A1
(43) Pub. Date: May 1, 2014

(54) METHOD AND APPARATUS FOR ASSISTING AIRWAY CLEARANCE

(71) Applicant: Christopher Wayne McDaniel, Bridgeville, PA (US)

(72) Inventor: Christopher Wayne McDaniel, Bridgeville, PA (US)

(73) Assignee: KONINKLIJKE PHILIPS N.V., EINDHOVEN (NL)

(21) Appl. No.: 14/125,717

(22) PCT Filed: Jun. 18, 2012
(86) PCT No.: PCT/IB2012/053048

§ 371 Date: Dec. 12, 2013

Related U.S. Application Data
(60) Provisional application No. 61/502,357, filed on Jun. 29, 2011.

(51) Int. Cl. A61M 16/00; A61M 16/04; A61M 16/08; A61M 16/06; A61M 16/20

(52) U.S. Cl. CPC A61M 16/0051; A61M 16/0066; A61M 16/06; A61M 16/20; A61M 16/0875; A61M 16/0465
USPC 128/204.23; 128/204.18; 128/205.25; 128/205.24

(57) ABSTRACT

A method of assisting a patient with airway clearance includes providing a positive pressure insufflation gas flow to the patient during an insufflation phase, following completion of the insufflation phase, causing the patient to enter an expiratory hold condition wherein the patient is prevented from exhaling, providing an abdominal thrust to the patient while the patient is in the expiratory hold condition, terminating the expiratory hold condition, and following termination of the expiratory hold condition, providing a negative pressure exsufflation gas flow to the patient during an exsufflation phase.

### CROSS-REFERENCE TO RELATED APPLICATIONS

This patent application claims the priority benefit under 35 U.S.C. §119(e) of U.S. Provisional Application No. 61/502,357 filed on Jun. 29, 2011, the contents of which are herein incorporated by reference.

### BACKGROUND OF THE INVENTION

1. Field of the Invention

The present invention pertains to a method and apparatus for assisting a subject in clearing his or her airway (i.e., coughing), and, in particular, to an in-exsufflation method and apparatus that includes an expiratory hold feature that is synchronized with a manually assisted cough.

2. Description of the Related Art

Coughing, also known as “airway clearance,” is a normal function of everyday life for most people. A typical cough sequence for a healthy individual able to cough normally is graphed in FIG. 1. As seen in FIG. 1, during an inspiratory phase, inhaled air is drawn into the lungs slowly (e.g., at a rate of less than 1 LPS) through the trachea. Then, during a compression phase, the individual's glottis, which covers the trachea, closes and the individual's expiratory muscles contract. As seen in FIG. 1, the closing of the glottis coupled with the contraction of the expiratory muscles increases the subglottic pressure (i.e., the pressure in the trachea below the closed glottis) of the individual. Finally, when the subglottic pressure reaches a certain level, the individual enters an expiratory phase of the cough. During the expiratory phase, the glottis opens and the initial flow outward is due to the decompression of the air in the trachea. Thereafter, the lungs continue to empty at a rate of roughly 4 LPS until the lungs are sufficiently decompressed.

Some people, due to injury, disease, or even thoracic surgery, have severely compromised or disabled glottic function, and, as a result, find it difficult or impossible to cough effectively on their own. This is largely due to the fact that such people are unable to increase their subglottic pressure during the compression phase of cough (FIG. 1) in preparation for the rapid decompression during the expiratory phase. For these people, assisted or artificial airway clearance is prescribed.

Artificial airway clearance can be achieved via many known methods. One such method employs the use of a device known as a mechanical in-exsufflator (MI-E). An MI-E is a medical device that delivers positive airway pressure through the mouth, nose, or a tracheostomy to gently fill the lungs to capacity (a process known as insufflation). It then very abruptly reverses pressure, which generates an explosive expiratory flow, mimicking a cough (a process known as exsufflation). Thus, MI-E devices attempt to generate effective expiratory flow rates through a combination of hyperinflation during the inspiratory phase and negative pressure during the expiratory phase.

In addition, in some cases, a physician or caregiver may wish to increase the effectiveness of the MI-E therapy by augmenting it with a manual-assist cough technique, such as a manually applied abdominal thrust. In such a case, the caregiver would skillfully position his or her hand(s) on the patient's abdomen and apply force in synch with the initiation of the exsufflation phase of the therapy (i.e., the force is applied in synch with the initiation and application of the reverse exsufflation pressure). This applied abdominal thrust will further increase the peak cough flow (PCF), and thus improve the effective mobilization of the patient's secretions.

While prior art artificial airway clearance methods as just described have been proven to be effective for many individual, there is room for improvement in this area. In particular, there is a need for an artificial airway clearance method that facilitates even higher PCF levels during treatment without requiring the MI-E device to deliver higher negative exsufflation pressures to the patient.

### SUMMARY OF THE INVENTION

Accordingly, it is an object of the present invention to provide a method of assisting a patient with airway clearance that overcomes the shortcomings of conventional airway clearance methods.

It is yet another object of the present invention to provide an apparatus for assisting a patient with airway clearance that does not suffer from the disadvantages associated with conventional devices techniques.

In one embodiment, a method of assisting a patient with airway clearance is provided that includes providing a positive pressure insufflation gas flow to the patient during an insufflation phase, following completion of the insufflation phase, causing the patient to enter an expiratory hold condition wherein the patient is prevented from exhaling, providing an abdominal thrust to the patient while the patient is in the expiratory hold condition, terminating the expiratory hold condition, and following termination of the expiratory hold condition, providing a negative pressure exsufflation gas flow to the patient during an exsufflation phase.

In another embodiment, an apparatus for assisting a patient with airway clearance is provided that includes a gas flow generating component structured to selectively generate a positive pressure insufflation gas flow and a negative pressure exsufflation gas flow, a patient circuit having a patient interface device operatively coupled to the gas flow generating component, and a controller operatively coupled to the gas flow generating component. The controller is structured to: (i) cause the gas flow generating component to provide the positive pressure insufflation gas flow to the patient through the patient circuit during an insufflation phase, (ii) determine that the insufflation phase is complete, (iii) responsive to determining that the insufflation phase is complete, cause the patient circuit to enter an operating condition wherein the patient, when coupled to the patient interface device, will be in an expiratory hold condition wherein the patient is prevented from exhaling, (iv) responsive to determining that a certain condition has been met, cause the patient circuit to no longer be in the operating condition such that the expiratory hold condition is terminated, and (v) following termination of the expiratory hold condition, cause the gas flow generating component to provide the negative pressure insufflation gas flow to the patient through the patient circuit during an exsufflation phase.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a graph illustrating a typical cough sequence in a healthy individual;

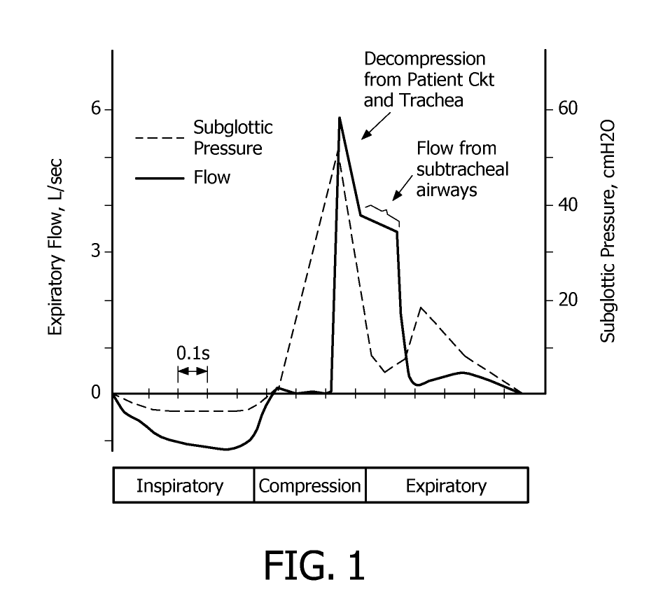

FIG. 2 is a schematic diagram of an MI-E device that may be used to implement an in-exsufflation method of the present invention according to one exemplary embodiment;

FIG. 3 is a schematic diagram of an MI-E device that may be used to implement an in-exsufflation method of the present invention according to an alternative exemplary embodiment;

FIG. 4 is a flowchart illustrating an in-exsufflation method according to an exemplary embodiment of the present invention;

FIG. 5 is a flowchart illustrating an in-exsufflation method according to one particular exemplary embodiment of the present invention;

FIG. 6 is a flowchart illustrating an in-exsufflation method according to an alternative particular exemplary embodiment of the present invention; and

FIGS. 7 and 8 are “idealized” graphs of time-based pressure and flow waveforms, respectively, that illustrate the benefits of the present invention.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

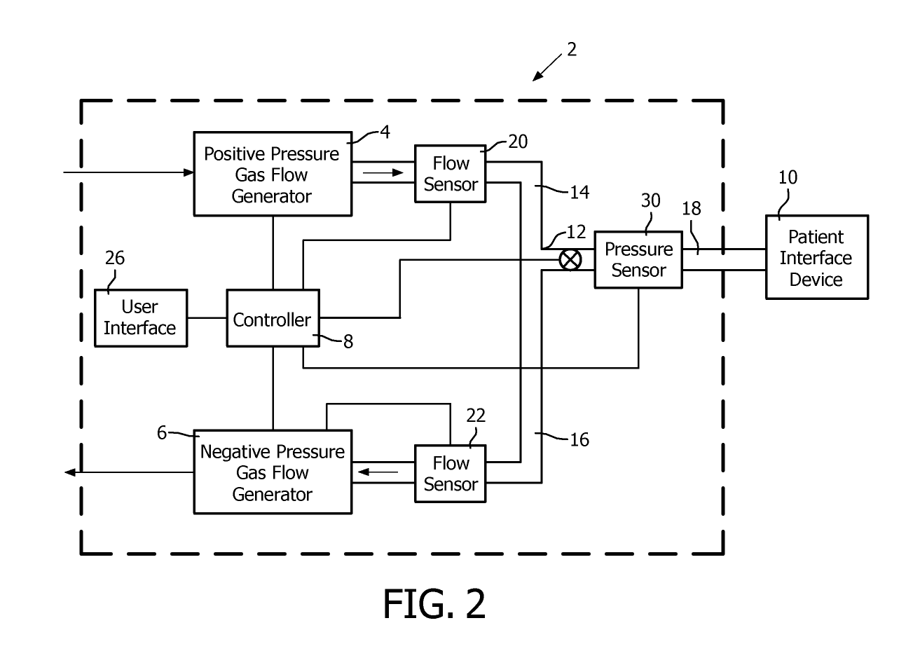

FIG. 2 is a schematic diagram of an MI-E device 2 that may be used to implement the in-exsufflation method of the present invention according to one exemplary embodiment. As seen in FIG. 2, MI-E device 2 includes a positive pressure gas flow generator 4 and a negative gas flow generator 6, each of which is operatively coupled to and controlled by a controller 8. Positive pressure gas flow generator 4 is structured to generate airflow under positive-pressure for use in insufflation of a patient as described herein. Positive pressure gas flow generator 4 may comprise a device such as a centrifugal blower (compressor), turbine, piston, bellows or another suitable apparatus known in the art for generating airflow under positive pressure. For example, positive pressure gas flow generator 4 may comprise, for example, a blower used in a conventional CPAP or bi-level pressure support device.

Negative pressure gas flow generator 6 is structured to generate airflow under negative-pressure for use in exsufflation of a patient as described herein. Negative pressure gas flow generator 6 may, like positive pressure gas flow generator 4, may comprise a device such as a centrifugal blower (compressor), turbine, piston, bellow or another suitable apparatus known in the art for generating airflow under negative pressure. In the exemplary embodiment, positive pressure gas flow generator 4 and negative pressure gas flow generator 6 each includes a valve (not shown) controlled by controller 8 that functions as a pressure controller or flow controller for positive pressure gas flow generator 4 and negative pressure gas flow generator 6 as the case may be.

It should be apparent that other techniques for controlling the pressure or the flow delivered by positive pressure gas flow generator 4 and negative pressure gas flow generator 6, such as varying the blower speed, either alone or in combination with a pressure control valve, are contemplated by the present invention. In addition, in use as described herein, when positive pressure gas flow generator 4 is providing a positive pressure flow, the valve associated with negative pressure gas flow generator 6 will be caused to be closed, and similarly, when negative pressure gas flow generator 6 is providing a negative pressure flow, the valve associated with positive pressure gas flow generator 4 will be caused to be closed.

Controller 8 includes a processing portion which may be, for example, a microprocessor, a microcontroller or some other suitable processing device, and a memory portion that may internal to the processing portion or operatively coupled to the processing portion and that provides a storage medium for data and software executable by the processing portion for controlling the operation of MI-e device 2 as described in greater detail herein.

MI-E device 2 also includes a patient interface device 10 that is coupled to both positive pressure gas flow generator 4 and negative pressure gas flow generator 6 by a Y-shaped delivery conduit 12 having a positive pressure branch 14 (connected to positive pressure gas flow generator 4), a negative pressure branch 16 (connected to negative pressure gas flow generator 6), and a common portion 18 (connected to patient interface device 10). Patient interface device 10, which may be a facemask, an endotracheal tube, a tracheostomy tube, or any other suitable means known in the art for establishing an interface between a patient and another medical device, interfaces positive pressure gas flow generator 4 and negative pressure gas flow generator 6 with a patient.

In the illustrated embodiment, a flow sensor 20 is provided in positive pressure branch 14 for measuring the gas flow rate therein and a flow sensor 22 is provided in negative pressure branch 16 for measuring the gas flow rate therein. The function of flow sensors 20, 22 in one particular, non-limiting exemplary embodiment is described elsewhere herein. As will be appreciated, in the event that that particular, non-limiting exemplary embodiment is not implemented, flow sensors 20, 22 may be omitted. Furthermore, in the illustrated embodiment, a valve 24, controlled by controller 8, is provided in common portion 18. Rather than being automatically controlled by controller 8, valve 24 may be manually controlled by a user of MI-E device 2, such as a clinician or caregiver. The function of valve 24 in one particular, non-limiting exemplary embodiment is described elsewhere herein. As will be appreciated, in the event that that particular, non-limiting exemplary embodiment is not implemented, valve 24 may be omitted.

MI-E device 2 also includes a user interface 26 for setting various parameters used by MI-E device 2, as well as for displaying and outputting information and data to a user, such as a clinician or caregiver.

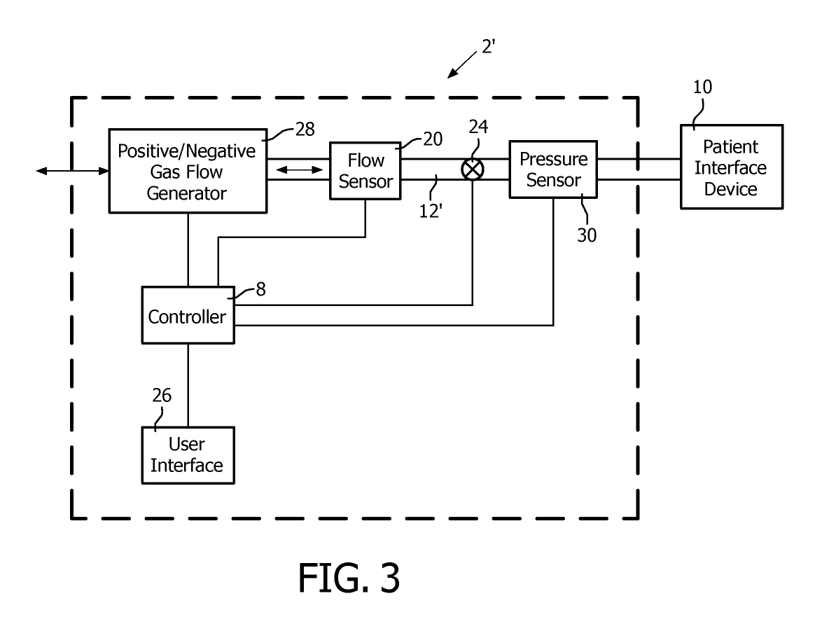

FIG. 3 is a schematic diagram of an MI-E device 2′ that may be used to implement the in-exsufflation method of the present invention according to an alternative exemplary embodiment. MI-E device 2′ includes many of the same components as MI-E device 2, and like parts are labeled with like reference numerals. However, as seen in FIG. 3, rather than including a separate positive pressure gas flow generator 4 and negative pressure gas flow generator 6, MI-E device 2′ includes a single component, positive/negative pressure gas flow generator 28, that is, under control of controller 8, structured to generate both airflow under positive-pressure for use in insufflation of a patient as described herein and airflow under negative-pressure for use in exsufflation of a patient as described herein. Positive/negative pressure gas flow generator 28 may comprise a device such as a centrifugal blower (compressor), turbine, piston, bellow or another suitable apparatus known in the art for selectively generating airflow under both positive and negative pressures. In the exemplary embodiment, positive/negative pressure gas flow generator 28 is a centrifugal blower that includes an arrangement of valves that is used to regulate the pressure that is delivered it to the patient. During the insufflation phase, the valves direct the outlet of the blower to the patient in order to deliver positive pressure. During the exsufflation phase, the valves direct the inlet side of the blower to the patient in order to deliver negative pressure.

According to an aspect of the present invention, the patient is placed in an expiratory hold condition. In that condition, the patient is prevented from exhaling through patient interface device 10. The patient's glottis may or may not close during the expiratory hold condition depending on the condition of the patient. In one embodiment, the expiratory hold condition is achieved by closing valve 24 such that the circuit including patient interface device 10 is substantially physically occluded. In another embodiment, the expiratory hold condition is achieved by increasing the positive supply pressure provided to the patient such that exhalation is inhibited. It will be understood that alternative manners and mechanisms for placing a patient in an expiratory hold condition are possible and contemplated within the scope of the present invention.

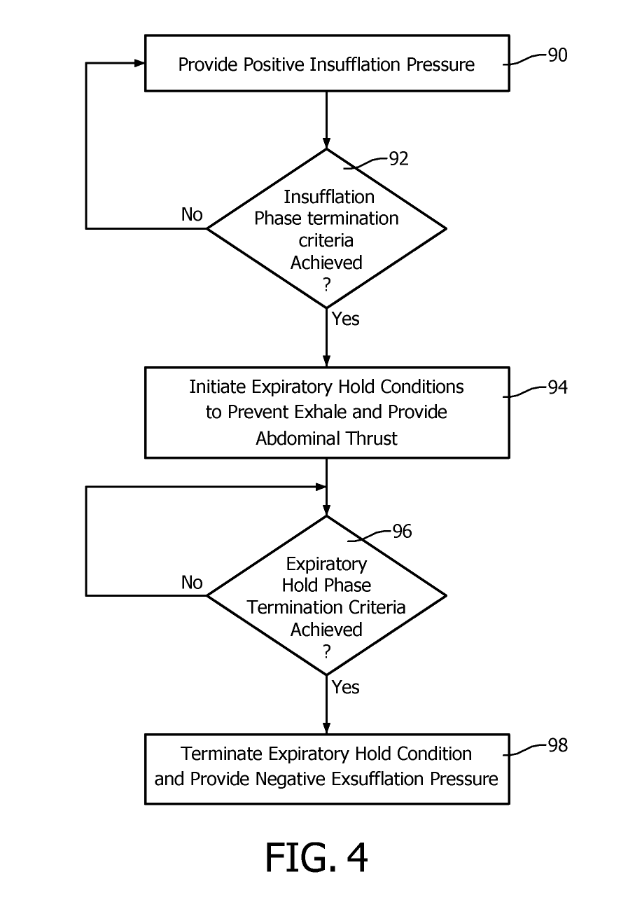

FIG. 4 is a flowchart illustrating an in-exsufflation method according to an exemplary embodiment of the present invention. The method shown in FIG. 4 may be performed using a suitable MI-E device, wherein certain portions of the method are implemented as one or more routines executable by the controller of the MI-E device for controlling the MI-E device as described. For purposes of illustrating the present invention, the method of FIG. 4 will be described as being implemented in either MI-E device 2 or MI-E device 2′.

Referring to FIG. 4, the method begins at step 90, wherein controller 8 causes a positive insufflation pressure to be provided to the patient (to inflate the patient's lungs) through patient interface device 10 by controlling either positive pressure gas flow generator 4 (in the case of MI-E device 2) or positive/negative pressure gas flow generator 28 (in the case of MI-E device 2′). Next, at step 92, a determination is made by controller 8 as to whether a predetermined insufflation phase termination criteria (e.g. a predetermined inflation volume, inflation pressure, inflation time, or inflation flow rate) has been achieved. If the answer at step 92 is no, then the method returns to step 90 and insufflation continues.

If, however, the answer at step 92 is yes, then, at step 94, controller 8 causes an expiratory hold condition, as described elsewhere herein, to be initiated. Also at step 94, during the expiratory hold condition, the patient's clinician or caregiver commences a manual abdominal thrust on the patient, which results in a rapid increase in the patient's subglottic pressure. In the exemplary embodiment, the commencement of the manual abdominal thrust is time synchronized with the successful establishment of the expiratory hold condition (i.e., it is commenced as soon as the expiratory hold condition is achieved). In one particular embodiment, controller 8 is structured to cause user interface 28 to provide a user perceptible indicator (e.g., an audible or visual indicator) once the expiratory hold condition is achieved to let the clinician or caregiver know that her or she can commence the abdominal thrust.

Next, at step 96, a determination is made by controller 8 as to whether a predetermined expiratory hold phase termination criteria (e.g., a time threshold, a pressure threshold or activation of a manual switch) has been achieved. If the answer at step 96 is no, then the method returns to step 96 and continues to wait for the predetermined expiratory hold phase termination criteria to be achieved. If the answer at step 96 is yes, meaning the predetermined expiratory hold phase termination criteria has been achieved, then the method proceeds to step 98.

At step 98, controller 8 terminates the expiratory hold condition (e.g., the increased positive pressure is removed or valve 24 is opened) and then causes a negative insufflation pressure to be provided to the patient (to deflate the patient's lungs) through patient interface device 10 by controlling either negative pressure gas flow generator 4 (in the case of MI-E device 2) or positive/negative pressure gas flow generator 28 (in the case of MI-E device 2′). According to an aspect of the present invention, because a higher subglottic pressure was generated as a result of the expiratory hold feature (combined with the abdominal thrust), a higher PCF will be achieved during the exsufflation phase (step 98). This higher PCF will in turn result in more effective secretion mobilization in the patient.

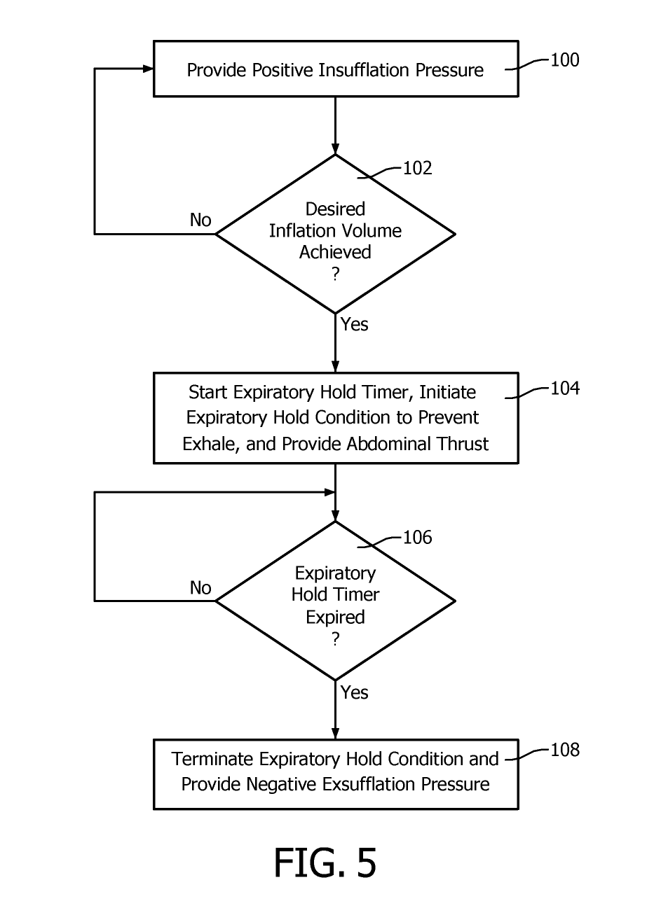

FIG. 5 is a flowchart illustrating an in-exsufflation method according to one particular exemplary embodiment of the present invention. The method shown in FIG. 5, like the method of FIG. 4, may be performed using a suitable MI-E device, wherein certain portions of the method are implemented as one or more routines executable by the controller of the MI-E device for controlling the MI-E device as described. For purposes of illustrating the present invention, the method of FIG. 5 will be described as being implemented in either MI-E device 2 or MI-E device 2′.

Referring to FIG. 5, the method begins at step 100, wherein controller 8 causes a positive insufflation pressure to be provided to the patient (to inflate the patient's lungs) through patient interface device 10 by controlling either positive pressure gas flow generator 4 (in the case of MI-E device 2) or positive/negative pressure gas flow generator 28 (in the case of MI-E device 2′). Next, at step 102, a determination is made by controller 8 as to whether a predetermined desired inflation volume of the patient's lungs has been achieved. In the exemplary embodiment, the actual inflation volume of the patient's lungs is determined based on flow measurements made by flow sensor 20 in a well known manner. The particular desired inflation volume may be set by a user, such as a clinician or caregiver, using user interface 26. In one exemplary, non-limiting embodiment, the desired inflation volume is the maximum insufflation capacity (MIC) of the patient. If the answer at step 102 is no, then the method returns to step 100 and insufflation continues.

If, however, the answer at step 102 is yes, then, at step 104, controller 8 does two things: (i) it starts a timer, referred to as an expiratory hold timer, and (ii) it causes an expiratory hold condition, as described elsewhere herein, to be initiated. The expiratory hold timer is a timer that determines how long (some predetermined fixed time period) the patient will be kept in the expiration hold condition. The particular duration of the expiratory hold timer may be set by a user, such as a clinician or caregiver, using user interface 26. In one exemplary, non-limiting embodiment, the duration of the expiratory hold timer is one second.

Also at step 104, during the expiratory hold condition, the patient's clinician or caregiver commences a manual abdominal thrust on the patient, which results in a rapid increase in the patient's subglottic pressure. In the exemplary embodiment, the commencement of the manual abdominal thrust is time synchronized with the successful establishment of the expiratory hold condition (i.e., it is commenced as soon as the expiratory hold condition is achieved). In one particular embodiment, controller 8 is structured to cause user interface 28 to provide a user perceptible indicator (e.g., an audible or visual indicator) once the expiratory hold condition is achieved to let the clinician or caregiver know that her or she can commence the abdominal thrust.

Next, at step 106, a determination is made by controller 8 as to whether the expiratory hold timer has expired. If the answer is no, then the method returns to step 106 and continues to wait for the expiratory hold timer to expire. If the answer at step 106 is yes, then the method proceeds to step 108. At step 108, controller 8 terminates the expiratory hold condition (e.g., the increased positive pressure is removed or valve 24 is opened) and then causes a negative insufflation pressure to be provided to the patient (to deflate the patient's lungs) through patient interface device 10 by controlling either negative pressure gas flow generator 4 (in the case of MI-E device 2) or positive/negative pressure gas flow generator 28 (in the case of MI-E device 2′). As described elsewhere herein, because a higher subglottic pressure was generated as a result of the expiratory hold feature (combined with the abdominal thrust), a higher PCF will be achieved during the exsufflation phase (step 108). This higher PCF will in turn result in more effective secretion mobilization in the patient.

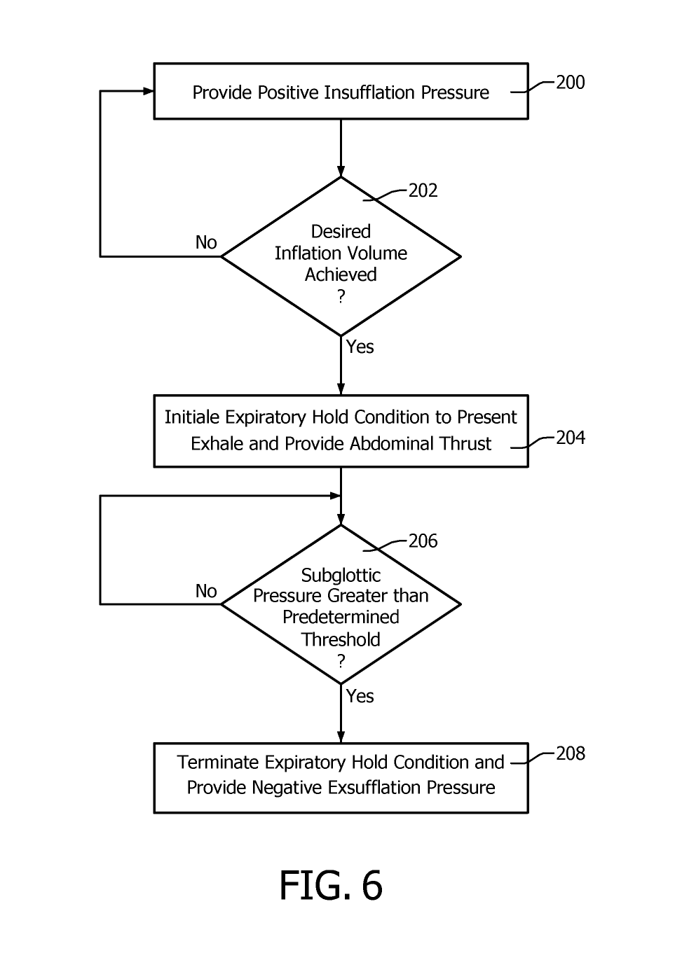

FIG. 6 is a flowchart illustrating an in-exsufflation method according to another particular exemplary embodiment of the present invention. The method shown in FIG. 6, like the methods of FIGS. 4 and 5, may be performed using a suitable MI-E device, wherein certain portions of the method are implemented as one or more routines executable by the controller of the MI-E device for controlling the MI-E device as described. For purposes of illustrating the present invention, the method of FIG. 6 will be described as being implemented in either MI-E device 2 or MI-E device 2′.

Referring to FIG. 6, the method begins at step 200, wherein controller 8 causes a positive insufflation pressure to be provided to the patient (to inflate the patient's lungs) through patient interface device 10 by controlling either positive pressure gas flow generator 4 (in the case of MI-E device 2) or positive/negative pressure gas flow generator 28 (in the case of MI-E device 2′). Next, at step 202, a determination is made by controller 8 as to whether a predetermined desired inflation volume, as described elsewhere herein, has been achieved. If the answer at step 202 is no, then the method returns to step 200 and insufflation continues.

If, however, the answer at step 202 is yes, then, at step 204, controller 8 causes an expiratory hold condition, as described elsewhere herein, to be initiated. As described in detail below, in the present embodiment, the duration of the expiratory hold condition is not a fixed time period (as was the case in the method of FIG. 5), but instead is determined based on the patient's subglottic pressure. Also at step 204, during the expiratory hold condition, the patient's clinician or caregiver commences a manual abdominal thrust on the patient, which results in a rapid increase in the patient's subglottic pressure. In the exemplary embodiment, the commencement of the manual abdominal thrust is time synchronized with the successful establishment of the expiratory hold condition (i.e., it is commenced as soon as the expiratory hold condition is achieved). As described elsewhere herein, in one particular embodiment, controller 8 is structured to cause user interface 28 to provide a user perceptible indicator (e.g., an audible or visual indicator) once the expiratory hold condition is achieved to let the clinician or caregiver know that her or she can commence the abdominal thrust.

Next, at step 206, a determination is made by controller 8 as to whether the patient's subglottic pressure has exceeded a predetermined threshold pressure level. In the exemplary embodiment, for this purpose, the patient's subglottic pressure is measured by a pressure sensor 30 that measures gauge pressure at the exit of MI-E device 2 or MI-E device 2′ (just before entering the patient tube connected to patient interface device 10). This sensor can be used to measure/calculate subglottic pressure with certain considerations in mind, depending on where you are in the time-based therapy profile. During the portions of the therapy when there is essentially zero flow, then the pressure measured by the sensor is arithmetically equivalent to the subglottic pressure in the patient's lungs. During the portions of the therapy when there is flow going through the system, the pressure measured by the sensor is a combination of subglottic pressure in the patient's lungs and the resistance of the patient circuit and patient airway structures leading up to the lungs. In this latter case, a mathematical model can be incorporated into the software algorithms, allowing the device to calculate subglottic pressure.

Referring again to step 206, the predetermined threshold pressure level may be set by a user, such as a clinician or caregiver, using user interface 26. In one exemplary, non-limiting embodiment, the predetermined threshold pressure level is 5 cmH2O above the pressure at the beginning of the expiratory hold phase. If the answer at step 206 is no, then the method returns to step 106 and continues to wait for the subglottic pressure to rise. If the answer at step 206 is yes, meaning the subglottic pressure has exceeded the predetermined threshold, then the method proceeds to step 208.

At step 208, controller 8 terminates the expiratory hold condition (e.g., the increased positive pressure is removed or valve 24 is opened) and then causes a negative insufflation pressure to be provided to the patient (to deflate the patient's lungs) through patient interface device 10 by controlling either negative pressure gas flow generator 4 (in the case of MI-E device 2) or positive/negative pressure gas flow generator 28 (in the case of MI-E device 2′). As described elsewhere herein, because a higher subglottic pressure was generated as a result of the expiratory hold feature (combined with the abdominal thrust), a higher PCF will be achieved during the exsufflation phase (step 208). This higher PCF will in turn result in more effective secretion mobilization in the patient.

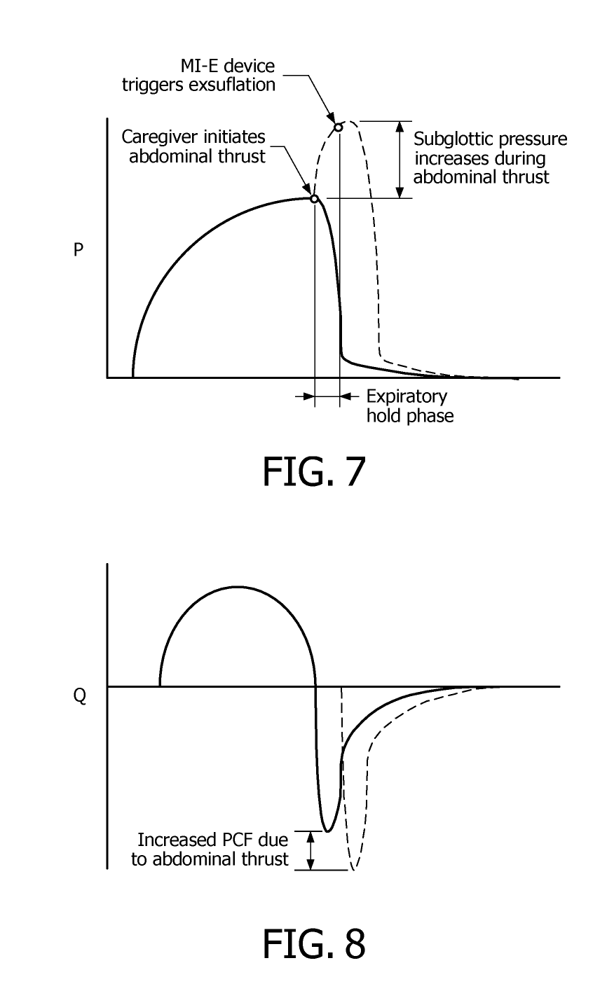

FIGS. 7 and 8 are “idealized” graphs of time-based pressure and flow waveforms, respectively, that illustrate the benefits of the present invention (solid lines are waveforms and generated based on use of prior art in-exsufflation methodology without an abdominal thrust and dotted lines are waveforms generated based on use of the methodology of the present invention). In FIG. 7, the pressure provided on the Y-axis is total pressure (subglottic+any flow resistance). Also in FIG. 7, in the prior art waveform (solid lines), the exsufflation phase begins at the point where the waveform starts heading back toward the x-axis. Note, the two waveforms in FIG. 7 are aligned with one another prior to the left of the first expiratory hold phase line.

In FIG. 8, the dotted line waveform of the invention is aligned with the prior art waveform up to the point that flow crosses the x-axis. Then, there is a period of time, during the abdominal thrust phase, when the dotted line waveform remains zero. Next, during the exsufflation phase, the dotted line waveform resumes a shape similar to that of the prior art. However, as result of the methodology of the invention, the dotted line waveform exhibits an increased peak (negative) flow. Thus, as seen in FIG. 7, the present invention provides increased subglottic pressure after insufflation as compared to the prior art, and as seen in FIG. 8, the present invention provides increased PCF after insufflation as compared to the prior art.

Furthermore, as described herein, flow sensors 20, 22 are positioned inside MI-E device 2 or MI-E device 2′, between the pressure/flow generator and the location of pressure sensor 30. The output of pressure sensor 30 is used by the software as the primary signal to control the therapy delivered by the device. However, in practice, this signal can be somewhat unstable, often resulting in premature, late, or even false triggering of the various phases of therapy. In one particular embodiment, by adding a flow signal from flow sensor 20 and/or 22 to the control algorithms, the system can often minimize the likelihood of this occurring. For example, if the system monitors a sudden drop in pressure, but the slope of the flow signal is negative, then the system can intelligently determine that it is not a good time to trigger a new breath. In an alternative embodiment, flow sensor 20, or 22 may be positioned inside MI-E device 2 or MI-E device 2′ between valve 24 and the location of pressure sensor 30.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A method of assisting a patient with airway clearance, comprising:
providing a positive pressure insufflation gas flow to the patient during an insufflation phase;
following completion of the insufflation phase, causing the patient to enter an expiratory hold condition wherein the patient is prevented from exhaling;
providing an abdominal thrust to the patient while the patient is in the expiratory hold condition;
terminating the expiratory hold condition; and
following termination of the expiratory hold condition, providing a negative pressure exsufflation gas flow to the patient during an exsufflation phase.

2. The method according to claim 1, wherein the insufflation phase continues until a certain inflation lung volume of the patient has been achieved.

3. The method according to claim 1, wherein the terminating the expiratory hold condition occurs a predetermined fixed amount of time after the patient has been caused to enter the expiratory hold condition.

4. The method according to claim 3, wherein a timer is started when the patient has been caused to enter the expiratory hold condition and wherein the terminating the expiratory hold condition occurs when the timer has indicated that the fixed amount of time has elapsed.

5. The method according to claim 1, wherein the causing the patient to enter an expiratory hold condition includes providing an increased positive pressure insufflation gas flow to the patient, the increased positive pressure insufflation gas flow having a pressure that is greater than a pressure of the positive pressure insufflation gas flow provided during the insufflation phase.

6. The method according to claim 1, wherein the causing the patient to enter an expiratory hold condition includes causing a patient circuit including a patient interface device through which the positive pressure insufflation gas flow is provided to the patient to be at least substantially physically occluded.

7. The method according to claim 6, wherein the causing the patient circuit to be at least substantially physically occluded comprises closing a valve provided in the patient circuit.

8. The method according to claim 1, further comprising determining a subglottic pressure of the patient while the patient is in the expiratory hold condition, wherein the terminating the expiratory hold condition occurs when the subglottic pressure exceeds a predetermined threshold level.

9. An apparatus for assisting a patient with airway clearance, comprising:
a gas flow generating component structured to selectively generate a positive pressure insufflation gas flow and a negative pressure exsufflation gas flow;
a patient circuit having a patient interface device operatively coupled to the gas flow generating component; and
a controller operatively coupled to the gas flow generating component, the controller being structured to: (i) cause the gas flow generating component to provide the positive pressure insufflation gas flow to the patient through the patient circuit during an insufflation phase, (ii) determine that the insufflation phase is complete, (iii) responsive to determining that the insufflation phase is complete, cause the patient circuit to enter an operating condition wherein the patient, when coupled to the patient interface device, will be in an expiratory hold condition wherein the patient is prevented from exhaling, (iv) responsive to determining that a certain condition has been met, cause the patient circuit to no longer be in the operating condition such that the expiratory hold condition is terminated, and (v) following termination of the expiratory hold condition, cause the gas flow generating component to provide the negative pressure insufflation gas flow to the patient through the patient circuit during an exsufflation phase.

10. The apparatus according to claim 9, further comprising a flow sensor provided in the patient circuit, wherein the controller is structured to measure an inflation lung volume of the patient based on an output of the sensor and that the insufflation phase is complete when the inflation lung volume reaches a certain level.

11. The apparatus according to claim 9, wherein the certain condition is a predetermined fixed amount of time elapsing after the patient circuit has been caused to enter the operating condition.

12. The apparatus according to claim 11, further comprising a pressure sensor structured to determine a subglottic pressure of the patient while the patient is in the expiratory hold condition, wherein certain condition is the subglottic pressure exceeding a predetermined threshold level.

13. The apparatus according to claim 9, wherein the causing the patient circuit to enter the operating condition includes causing the gas flow generating component to provide an increased positive pressure insufflation gas flow to the patient, the increased positive pressure insufflation gas flow having a pressure that is greater than a pressure of the positive pressure insufflation gas flow provided during the insufflation phase.

14. The apparatus according to claim 9, further comprising a valve provided in the patient circuit, wherein the causing the patient circuit to enter the operating condition includes closing the valve to causing the patient circuit to be at least substantially physically occluded.

15. The apparatus according to claim 9, wherein the gas flow generating component comprises a positive pressure gas flow generator structured to generate the positive pressure insufflation gas flow and a separate negative pressure gas flow generator structured to generate the negative pressure exsufflation gas flow.

16. The apparatus according to claim 9, wherein the gas flow generating component comprises a gas flow generator structured to generate both the positive pressure insufflation gas flow and the negative pressure exsufflation gas flow.
